(19) United States
(12) Patent Application Publication
(10) Pub. No.: US 2024/0221869 A1
(43) Pub. Date: Jul. 4, 2024

(54) METHOD AND SYSTEM FOR CONVERTING A PROTEIN DATA BANK FILE INTO A TWO-DIMENSIONAL NUMERICAL MATRIX

(71) Applicant: Innoplexus AG, Eschborn (DE)

(72) Inventors: Sudhanshu Kumar, Bokaro (IN); Joel Joseph, Palayi (IN); Ansh Gupta, Menhdawal (IN)

(73) Assignee: Innoplexus AG, Eschborn (DE)

(21) Appl. No.: 18/148,794

(22) Filed: Dec. 30, 2022

(51) Int. Cl. G16B 50/00; G06F 16/16; G16B 30/00

(52) U.S. Cl. CPC G16B 50/00; G06F 16/16; G16B 30/00

(57) ABSTRACT

A system and a method for converting a protein data bank file into a two-dimensional numerical matrix is provided. The method also includes extracting PDB files from a PDB, using a data extraction module. The method further includes analyzing the PDB files using an analysis module for calculating and visualizing interatomic interactions in protein structures. The method further includes generating, using a file generation module, a comma separated values (CSV) file based on the analysis. The method further includes performing a featurization of the CSV file using a featurization module. The method further includes generating, using a matrix generating module, the two-dimensional numerical matrix based on the featurization.

### BACKGROUND

### Technical Field

The present invention is generally related to the field of protein engineering. More particularly, the present invention is related to a method and system for converting a protein data bank file into a two-dimensional numerical matrix.

### Description of the Related Art

Generally, protein engineering involves the development of proteins that have certain biological activities. Antibodies are proteins which are usually generated by the body of organisms to defeat foreign agents, usually other protein structures called Antigens. With the enhancement of biomolecule development in the laboratory setting, techniques have been developed to generate artificial antibodies that can deal with specific antigens and also measure their activity towards those antigens. Application of machine learning in antibody optimization has led to the requirement of easily processable numeric encodings or embeddings. Generating features from protein data bank (PDB) files has increasingly become one of the major goals of biotechnology and biomedicine over the past few decades. Typically, a PDB file is a docked structure of antibody-antigen compounds which could be visualized as a three-dimensional (3-D) structure. However, in PDB files, the data is present in a format that cannot be directly processed by artificial intelligence models, for a featurizer to efficiently featurize a PDB file to a set of numeric values. Additionally, given a PDB file, it is a tedious task to generate promising features consisting of only numbers without loss of much information.

Several existing techniques, such as Onionnet create features from PDB files using their shell-based technique. They first calculate the distance between atoms of protein and ligand and then create imaginary shells of fixed radius around protein atoms and determine points of contact with the ligand atoms giving a binary value 0 or 1 for each contact. They create multiple shells of consecutively increasing sizes for generating the features. This technique is used only for PDBs of protein ligand complexes. Techniques such as Prodigy predict binding affinity values for protein-protein complexes from atomic structures. They compute a number of intermolecular contacts, no of charged-charged contacts, no of charged-polar contacts, no of charged-apolar contacts, no of polar-polar contacts, no of apolar-polar contacts, no of apolar-apolar contacts, percentage of apolar NIS residues and percentage of charged NIS residues from the PDB file. They then generate some weightages to these metrics by fitting the linear regression model and calculate the binding affinity using the above features. The above technique is not very robust as it uses very few features (only contact based features) and also uses a simple linear regression model which oversimplifies the problem by assuming linear relationship among features which leads to poor prediction in majority of cases. However, none of the known techniques have an efficient way of conversion of the PDB files into in a format that can be directly processed by artificial intelligence models, for a featurizer to efficiently featurize a PDB file to a set of numeric values. Hence there is a need for a method and system for converting a protein data bank file into a two-dimensional numerical matrix.

The above-mentioned shortcomings, disadvantages and problems are addressed herein, and will be understood by reading and studying the following specification.

### SUMMARY

This summary is provided to introduce a selection of concepts in a simplified form that are further disclosed in the detailed description. This summary is not intended to determine the scope of the claimed subject matter.

The embodiments herein address the above-recited needs for a system and a method for converting PDB file into a two-dimensional numerical matrix. The method and system of the present technology for generating the two-dimensional numerical matrix from the PDB files increases efficiency in binding affinity prediction and also enables faster development of drugs. Using the method and system of the present technology HER2 antibodies (Drugs) can be generated which are more effective at neutralizing the HER2 antigen.

According to one aspect, a processor implemented method of converting protein data bank (PDB) files into a two-dimensional numerical matrix is provided. The method includes extracting PDB files from a PDB. The method also includes analyzing the PDB files for calculating and visualizing interatomic interactions in protein structures. The method further includes generating a comma separated values (CSV) file based on the analysis. The method furthermore includes performing a featurization of the CSV file. The method furthermore includes generating, using a matrix generating module, the two-dimensional numerical matrix based on the featurization.

In an embodiment, performing the featurization includes positioning a distance weightage of amino acids in the two-dimensional numerical matrix next to a predetermined row and a predetermined column upon on a predetermine criterion being satisfied.

In an embodiment, generating the CSV file based on the analysis includes receiving a docked antibody-antigen complex file as input and generating the CSV file including a plurality of amino acids with respective bond distances.

According to another aspect, a system for converting a protein data bank file into a two-dimensional numerical matrix is disclosed. The system includes a processor configured to execute non-transitory machine-readable instructions that when executed causes the processor to extract PDB files from a PDB; analyze the PDB files for calculating and visualizing interatomic interactions in protein structures; generate a comma separated values (CSV) file based on the analysis; perform a featurization of the CSV file; and generate using the two-dimensional numerical matrix based on the featurization.

In an embodiment, the processor is further configured to position a distance weightage of amino acids in the two-dimensional numerical matrix next to a predetermined row and a predetermined column upon a predetermined criterion being satisfied.

In an embodiment, the processor is further configured to receive a docked antibody-antigen complex file as input and generate the CSV file including a plurality of amino acids with respective bond distances.

According to yet another aspect, one or more non-transitory computer readable storage mediums storing one or more sequences of instructions, which when executed by one or more processors, causes a method for converting a protein data bank file into a two-dimensional numerical matrix. The method includes extracting PDB files from a PDB. The method also includes analyzing the PDB files for calculating and visualizing interatomic interactions in protein structures. The method further includes generating a comma separated values (CSV) file based on the analysis. The method furthermore includes performing a featurization of the CSV file. The method furthermore includes generating the two-dimensional numerical matrix based on the featurization.

The method and system of the present technology provides an efficient technique for converting protein data bank (PDB) files into a two-dimensional numerical matrix that in turn reduces processing complexity and improves efficiency of processes the PDB files are subjected to in protein engineering such as featurization of PDB files. The present technology is extremely useful for the faster development of drugs such as HER2. Antibodies (Drugs) can be generated which are more effective at neutralizing the HER2 antigen. The present technology is also useful in other antibody optimization tasks in the bioinformatics domain. The present technology also helps in featurization of the PDB file. The method and system convert the PDB file into a format that will contain more concise information that could be directly seen by machine learning models.

It is to be understood that the aspects and embodiments of the disclosure described above may be used in any combination with each other. Several of the aspects and embodiments may be combined to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

These and other objects and advantages will become more apparent when reference is made to the following description and accompanying drawings.

### BRIEF DESCRIPTION OF THE DRAWINGS

The other objects, features and advantages will occur to those skilled in the art from the following description of the preferred embodiment and the accompanying drawings in which:

FIG. 1 depicts an architecture of an implementation of system for converting protein data bank (PDB) files into a two-dimensional numerical matrix, according to one or more embodiments;

FIG. 2 depicts a sample PDB file that the Arpeggio tool takes as input, in accordance with an exemplary scenario;

FIG. 3 depicts a sample comma separated values (CSV) file generated from the PDB file, in accordance with an exemplary scenario;

FIG. 4 depicts an example two-dimensional matrix generated form the CSV file by the system of the present technology, in accordance with an exemplary scenario;

FIG. 5 illustrates a flow diagram depicting a method of converting protein data bank (PDB) files into a two-dimensional matrix; and

FIG. 6 is a computer system used in accordance with the embodiments herein.

Although the specific features of the embodiments herein are shown in some drawings and not in others. This is done for convenience only as each feature may be combined with any or all of the other features in accordance with the embodiments herein.

### DETAILED DESCRIPTION OF THE DRAWINGS

The detailed description of various exemplary embodiments of the disclosure is described herein with reference to the accompanying drawings. It should be noted that the embodiments are described herein in such details as to clearly communicate the disclosure. However, the amount of details provided herein is not intended to limit the anticipated variations of embodiments; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail below. It should be understood, however, that it is not intended to limit the disclosure to the forms disclosed, but on the contrary, the disclosure is to cover all modifications, equivalents, and alternatives falling within the scope of the disclosure.

The detailed description of various exemplary embodiments of the disclosure is described herein with reference to the accompanying drawings. It should be noted that the embodiments are described herein in such details as to clearly communicate the disclosure. However, the details provided herein is not intended to limit the anticipated variations of embodiments; on the contrary, the intention is to cover all modifications, equivalents, and alternatives falling within the spirit and scope of the present disclosure as defined by the appended claims.

It is also to be understood that various arrangements may be devised that, although not explicitly described or shown herein, embody the principles of the present disclosure. Moreover, all statements herein reciting principles, aspects, and embodiments of the present disclosure, as well as specific examples, are intended to encompass equivalents thereof.

While the disclosure is susceptible to various modifications and alternative forms, specific embodiment thereof has been shown by way of example in the drawings and will be described in detail below. It should be understood however, it is not intended to limit the disclosure to the forms disclosed, but on the contrary, the disclosure is to cover all modifications, equivalents, and alternatives falling within the scope of the disclosure.

The various embodiments of the present technology provides an efficient technique for generating a two-dimensional numerical matrix from protein data bank files that could be fed into a machine learning model for a wide range of tasks. The present technology provides a method and system for converting protein data bank (PDB) files into a two-dimensional numerical matrix. In an embodiment, the method and system of the present technology can be used for conversion of the PDB files into in a format that can be directly processed by artificial intelligence models, for a featurizer to efficiently featurize a PDB file to a set of numeric values, that can be applied for various applications such as computation of a binding affinity between antibody and antigen molecules based on deep learning. Typically, the three-dimensional (3D) structure of protein-protein complexes is stored in PDB files. The PDB files stores the x, y, z coordinates of atoms in the molecule along with the chain name, and other information. The information in the PDB files cannot be directly fed into neural network/other artificial intelligence models and hence need to be converted into a usable format using different types of featurization techniques The present technology can be used for efficiently convert the PDB files into a format that can be easily processed in the featurizer to generate artificial intelligence models based on deep learning and to process and classify protein sequences of antigens and antibodies in the scale of more than 1 million sequences for computing the binding affinity of the protein sequences, the information associated with which can be later used for drug development and discovery applications.

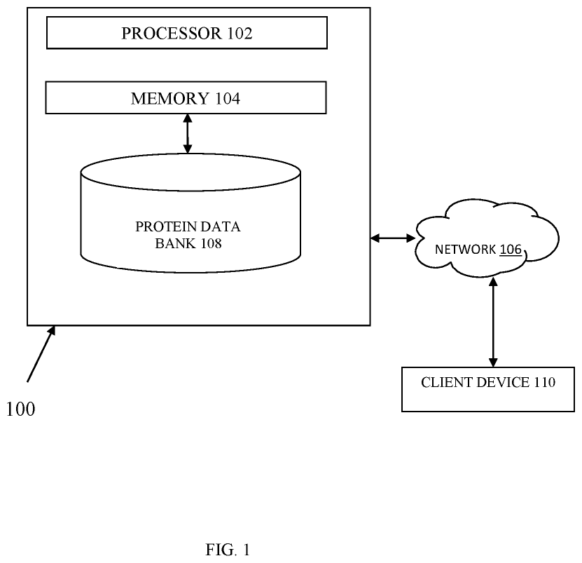

Referring to FIG. 1, the system 100 comprises a processor 102 with a memory 104 comprising executable non-transitory machine-readable instructions wherein the processor 102 is configured to execute the said machine-readable instructions. The system 100 includes a processor 102 and a memory 104. The processor 102 is communicably coupled to a client device 110 via a data communication network 106. The network 106 may be for example, a private network and a public network, a wired network or a wireless network. The wired network may include, for example Digital Subscriber Line (DSL), coaxial cable lines, or optical fiber lines. The wireless network may include for example Bluetooth®, Bluetooth Low Energy (BLE), ANT/ANT+, ZigBee, Z-Wave, Thread, Wi-Fi®, Worldwide Interoperability for Microwave Access (WiMAX®), mobile WiMAX®, WiMAX®-Advanced, a satellite band and other similar wireless networks. The wireless networks may also include any cellular network standards to communicate among mobile devices. Examples of the client device 110 includes but is not limited to user devices (such as cellular phones, personal digital assistants (PDAs), handheld devices, laptop computers, personal computers, an Internet-of-Things (IOT) device, a smart phone, a machine type communication (MTC) device, a computing device, a drone, or any other portable or non-portable electronic device. According to some embodiments, the system 100 may be implemented in a variety of computing systems, such as a mainframe computer, a server, a network server, a laptop computer, a desktop computer, a notebook, a workstation, and the like. In an implementation, the system 100 may be implemented in a server or in a computing device. In some embodiments, the system 100 may be implemented as a part of a cluster of servers. In some embodiments, the system 100 may be performed by the plurality of servers. These tasks may be allocated among the cluster of servers by an application, a service, a daemon, a routine, or other executable logic for task allocation.

The system 100 also includes a protein data bank 108 communicably coupled to the memory 104. The processor 102 is configured to extract PDB files from a PDB, using a data extraction module. The PDB 108 stores data associated with all protein complexes, such as three-dimensional (3D) structure of the protein complexes, PDB index of the protein complexes, residue range, chain IDs, and the like. As used herein the term “PDB” stands for a database for a three-dimensional structural data of large biological molecules, such as proteins and nucleic acids. In the PDB data file format for macromolecular models, each atom is designated either ATOM or HETA™ (which stands for hetero atom). ATOM is reserved for atoms in standard residues of protein, DNA or RNA. HETA™ is applied to non-standard residues of protein, DNA or RNA, as well as atoms in other kinds of groups, such as carbohydrates, substrates, ligands, solvent, and metal ions.

According to some embodiments, the processor 102 is configured to analyze the PDB files for calculating and visualizing interatomic interactions in protein structures. In an embodiment, the analysis is performed using an Arpeggio tool. The Arpeggio tool provides more direct information about atomic binding (pairs of binding atoms, distance, and type of contact between them) for extracting useful binding information from the PDB file compared to other techniques. Additionally, using Arpeggio, the system 100 provides accurate information about binding to the machine learning (ML) model, so that the ML models can be trained effectively. In several other embodiments, other web tools that can calculate, visualise, and understand the interactions in protein structures may be used. Interactions between proteins, such as small molecules, other proteins, and DNA, depend on specific interatomic interactions that can be classified on the basis of atom type and distance and angle constraints. Visualisation of these interactions provides insights into the nature of molecular recognition events and has practical uses in guiding drug design and understanding the structural and functional impacts of mutations. The processor is configured to generate a comma separated values (CSV) file based on the analysis. In some embodiments, in order to generate the CSV file, the processor receives a docked antibody-antigen complex file as input and generates the CSV file including a plurality of amino acids with respective bond distances. The PDB file is loaded into a data frame and using the data frame, the PDB file is converted into the CSV file using the python package. Docking is a process of predicting the structure of a multi-molecular complex from the structures of its separated components. In a typical docking protocol, the structures of the antigen and antibody are separated by approximately 25 Å and subsequently brought together by a chosen algorithm. The docked PDB files are used to generate the CSV file.

In an embodiment, Arpeggio tool is used to generate CSV file from PDB files. Arpeggio is a web server for calculating interactions within and between proteins and protein, DNA, or small-molecule ligands, including van der Waals', ionic, carbonyl, metal, hydrophobic, and halogen bond contacts, and hydrogen bonds and specific atom-aromatic ring (cation-π, donor-π, halogen-π, and carbon-π) and aromatic ring-aromatic ring (x-x) interactions, within user-submitted macromolecule structures.

In an embodiment, the docked PDB file structure is sent to a pigeon file. The pigeon file generates columns which are not relevant to us. The relevant columns are shortlisted, including begin label and end label columns and there are the atoms binding together. The distance between these relevant columns is used to determine the weightage. If distance is small a higher weightage is provided, as the binding is strong. If the distance between the molecules are large then a smaller weightage is given.

According to some embodiments, the processor is configured to perform a featurization of the CSV file. In an embodiment, the processor positions a distance weightage of amino acids in the two-dimensional numerical matrix next to a predetermined row and a predetermined column upon on a predetermine criterion being satisfied. In an embodiment, the processor generates two-dimensional numerical matrix based on the featurization. In an embodiment, the two-dimensional numerical matrix includes an adjacency matrix. In an embodiment, the processor takes the CSV file as input and generates a distance weighted adjacency matrix by considering three columns in the CS file, including bgn_amino_acid, end_amino_acid, distance_weightage columns. The processor names all the rows of the adjacency matrix as the 21 existing amino acids and similarly names all the columns of the adjacency matrix also as the 21 existing amino acids. Then in the csv file, if a bgn_amino_acid is interacting with the end_amino_acid then the processor puts the distance weightage of those amino acids in the adjacency matrix in front of that particular row and column. In this way, the processor generates a sufficiently small adjacency matrix of size 21*21 only, containing most of the useful information of the PDB file for binding affinity prediction.

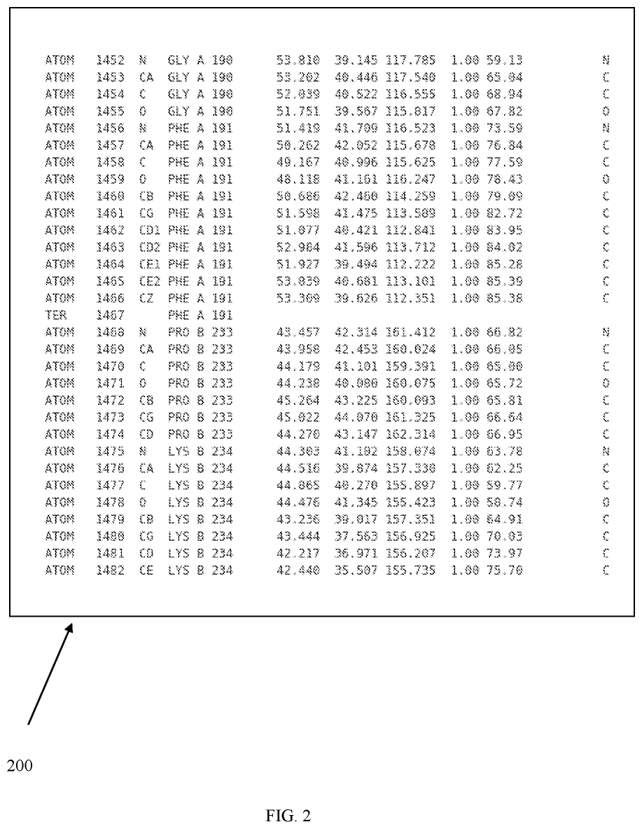

FIG. 2 depicts a sample PDB file 200 that the Arpeggio tool takes as input, in accordance with an exemplary scenario. As shown in FIG. 2, is suppose there are 21 unique amino acids present, the processor considers 21 rows and 21 columns corresponding to amino acids. The processor maps each amino acid with other. Diagonally the distance is zero. ALA-ALA is zero. Last row CYR-CYS the distance weightage is 3.125. The processor generates the CSV file using the PDB file.

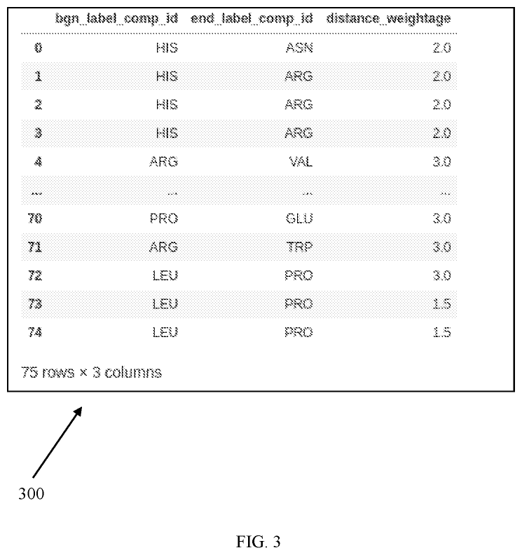

FIG. 3 depicts a sample comma separated values (CSV) file 300 generated from the PDB file, in accordance with an exemplary scenario;

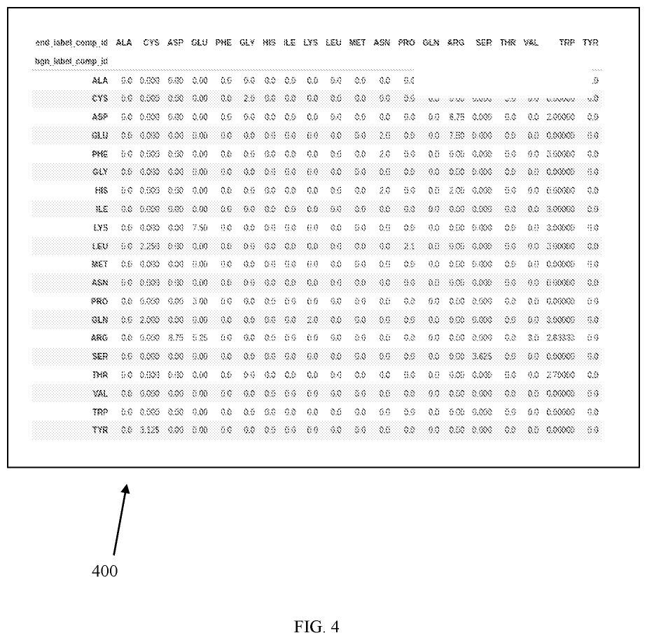

FIG. 4 depicts an example two-dimensional matrix 400 generated form the CSV file by the system of the present technology, in accordance with an exemplary scenario;

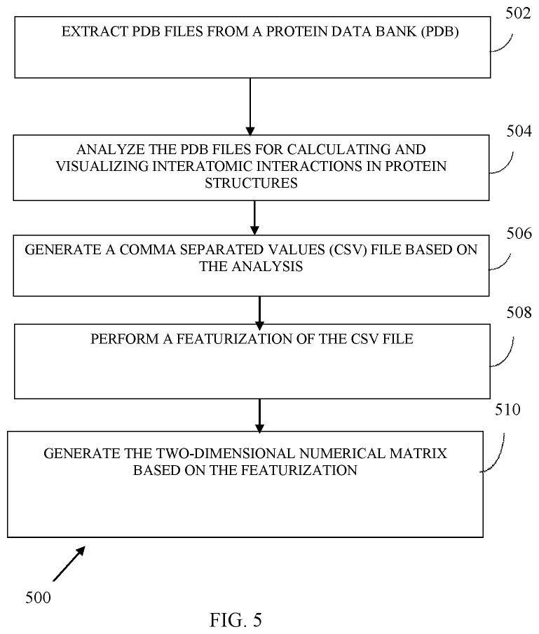

FIG. 5 illustrates a flow diagram 500 depicting a method of converting protein data bank (PDB) files into a two-dimensional matrix. At step 502, files from a PDB file are extracted. At step 504, the PDB files are analyzed for calculating and visualizing interatomic interactions in protein structures. At step 506, a comma separated values (CSV) file is generated based on the analysis. At step 508, a featurization of the CSV file is performed. At step 510, a two-dimensional numerical matrix based on the featurization.

In an embodiment, performing the featurization includes positioning a distance weightage of amino acids in the two-dimensional numerical matrix next to a predetermined row and a predetermined column upon on a predetermine criterion being satisfied.

In an embodiment, generating the CSV file based on the analysis includes receiving a docked antibody-antigen complex file as input and generating the CSV file including a plurality of amino acids with respective bond distances.

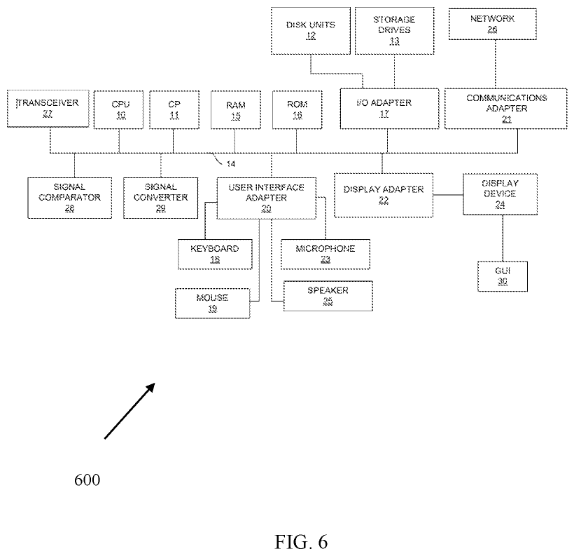

A representative hardware environment for practicing the embodiments herein is depicted in FIG. 6 with reference to FIGS. 1 through 5. This schematic drawing illustrates a hardware configuration of system 100 of FIG. 1, in accordance with the embodiments herein. The hardware configuration includes at least one processing device 10 and a cryptographic processor 11. The computer system 104 may include one or more of a personal computer, a laptop, a tablet device, a smartphone, a mobile communication device, a personal digital assistant, or any other such computing device, in one example embodiment. The computer system 104 includes one or more processor (e.g., the processor 108) or central processing unit (CPU) 10. The CPUs 10 are interconnected via system bus 12 to various devices such as a memory 14, read-only memory (ROM) 16, and an input/output (I/O) adapter 18. Although CPUs 10 are depicted, it is to be understood that the computer system 104 may be implemented with only one CPU.

The I/O adapter 18 can connect to peripheral devices, such as disk units 11 and tape drives 13, or other program storage devices that are readable by the system. The computer system 104 can read the inventive instructions on the program storage devices and follow these instructions to execute the methodology of the embodiments herein. The computer system 104 further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

Various embodiments of the present technology provides an efficient technique for converting protein data bank (PDB) files into a two-dimensional matrix that in turn reduces processing complexity and improves efficiency of processes the PDB files are subjected to in protein engineering such as featurization of PDB files. The present technology is extremely useful for the faster development of drugs such as HER2. Antibodies (Drugs) can be generated which are more effective at neutralizing the HER2 antigen. The present technology is also useful in other antibody optimization tasks in the bioinformatics domain.

The embodiments herein can include both hardware and software elements. The embodiments that are implemented in software include but are not limited to, firmware, resident software, microcode, etc. Furthermore, the embodiments herein can take the form of a computer program product accessible from a computer-usable or computer-readable medium providing program code for use by or in connection with a computer or any instruction execution system. For the purposes of this description, a computer-usable or computer readable medium can be any apparatus that can comprise, store, communicate, propagate, or transport the program for use by or in connection with the instruction execution system, apparatus, or device.

The system, method, computer program product, and propagated signal described in this application may, of course, be embodied in hardware; e.g., within or coupled to a Central Processing Unit (“CPU”), microprocessor, microcontroller, System on Chip (“SOC”), or any other programmable device. Additionally, the system, method, computer program product, and propagated signal may be embodied in software (e.g., computer readable code, program code, instructions and/or data disposed in any form, such as source, object or machine language) disposed, for example, in a computer usable (e.g., readable) medium configured to store the software. Such software enables the function, fabrication, modeling, simulation, description and/or testing of the apparatus and processes described herein.

Such software can be disposed in any known computer usable medium including semiconductor, magnetic disk, optical disc (e.g., CD-ROM, DVD-ROM, and the like) and as a computer data signal embodied in a computer usable (e.g., readable) transmission medium (e.g., carrier wave or any other medium including digital, optical, or analog-based medium). As such, the software can be transmitted over communication networks including the Internet and intranets. A system, method, computer program product, and propagated signal embodied in software may be included in a semiconductor intellectual property core (e.g., embodied in HDL) and transformed to hardware in the production of integrated circuits. Additionally, a system, method, computer program product, and propagated signal as described herein may be embodied as a combination of hardware and software

A “computer-readable medium” for purposes of embodiments of the present invention may be any medium that can contain, store, communicate, propagate, or transport the program for use by or in connection with the instruction execution system, apparatus, system or device. The computer readable medium can be, by way of example only but not by limitation, an electronic, magnetic, optical, electromagnetic, infrared, or semiconductor system, apparatus, system, device, propagation medium, or computer memory.

A “processor” or “process” includes any human, hardware and/or software system, mechanism or component that processes data, signals or other information. A processor can include a system with a general-purpose central processing unit, multiple processing units, dedicated circuitry for achieving functionality, or other systems. Processing need not be limited to a geographic location or have temporal limitations. For example, a processor can perform its functions in “real time,” “offline,” in a “batch mode,” etc. Portions of processing can be performed at different times and at different locations, by different (or the same) processing systems.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims. The scope of the embodiments will be ascertained by the claims to be submitted at the time of filing a complete specification.

What is claimed is:

1. A processor-implemented method of converting a protein data bank file into a two-dimensional numerical matrix, the method comprising:
extracting PDB files from a PDB;
analyzing the PDB files for calculating and visualizing interatomic interactions in protein structures;
generating a comma separated values (CSV) file based on the analysis;
performing a featurization of the CSV file; and
generating the two-dimensional numerical matrix based on the featurization.

2. The processor-implemented method of claim 1, wherein performing the featurization comprises:
positioning a distance weightage of amino acids in the two-dimensional numerical matrix next to a predetermined row and a predetermined column upon on a predetermine criterion being satisfied.

3. The processor-implemented method of claim 1, wherein generating a CSV file based on the analysis comprises:
receiving a docked antibody-antigen complex file as input; and
generating the CSV file including a plurality of amino acids with respective bond distances.

4. A system for converting a protein data bank file into a two-dimensional numerical matrix, the system comprising a processor configured to execute non-transitory machine-readable instructions that when executed causes the processor to:
extract PDB files from a PDB;
analyze the PDB files for calculating and visualizing interatomic interactions in protein structures;
generate a comma separated values (CSV) file based on the analysis;
perform a featurization of the CSV file; and
generate using the two-dimensional numerical matrix based on the featurization.

5. The system of claim 4, wherein the processor is further configured to:
position a distance weightage of amino acids in the two-dimensional numerical matrix next to a predetermined row and a predetermined column upon on a predetermine criterion being satisfied.

6. The system of claim 4, wherein the processor is further configured to:
receive a docked antibody-antigen complex file as input; and
generate the CSV file including a plurality of amino acids with respective bond distances.

7. One or more non-transitory computer readable storage mediums storing one or more sequences of instructions, which when executed by one or more processors, causes a method for converting a protein data bank file into a two-dimensional numerical matrix, the method comprising:
extracting PDB files from a PDB;
analyzing the PDB files for calculating and visualizing interatomic interactions in protein structures;
generating a comma separated values (CSV) file based on the analysis;
performing a featurization of the CSV file; and
generating the two-dimensional numerical matrix based on the featurization.
